(19) United States
(12) Patent Application Publication
(10) Pub. No.: US 2016/0206717 A1
(43) Pub. Date: Jul. 21, 2016

(54) STIMULATION OF IMMUNITY TO ENDOTHELIAL CELLS, ENDOTHELIAL-LIKE CELLS, AND INTRATUMOR VASCULAR CHANNELS DERIVED FROM TUMOR TISSUE

(71) Applicant: Batu Biologics, Inc., San Diego, CA (US)

(72) Inventors: Samuel C. Wagner, San Diego, CA (US); Thomas E. Ichim, San Diego, CA (US); Santosh Kesari, San Diego, CA (US); Amit N. Patel, Salt Lake City, UT (US)

(21) Appl. No.: 15/001,161

(22) Filed: Jan. 19, 2016

Related U.S. Application Data
(60) Provisional application No. 62/104,123, filed on Jan. 16, 2015.

(51) Int. Cl. A61K 39/00; C12N 5/0786

(52) U.S. Cl. CPC A61K 39/0011; C12N 5/0645; C12N 2501/2304; C12N 2501/2313; A61K 2039/5152

(57) ABSTRACT

Disclosed are compositions of matter, methods, and protocols useful for treatment of cancer through induction of anti-angiogenic immune responses. The invention provides means of differentiating tumor cells directly into endothelial or endothelial-like cells and utilizing said cells as immunogens for the purpose of inducing immunity against blood vessels feeding tumors. In one embodiment glioma cells are cultured under hypoxic conditions in the presence of endothelial-differentiating factors. In another embodiment, PECAM-1 positive cells are derived from a tumor mass or cell line and utilized as an antigenic source to induce immunity towards tumor derived endothelial cells, endothelial-like cells, and tumor vascular channels.

### CROSS-REFERENCE TO RELATED APPLICATION

This application claims the benefit of U.S. Provisional Application No. 62/104,123 filed on Jan. 16, 2015, the contents of which are incorporated herein by reference in its entirety.

### FIELD OF THE INVENTION

The invention pertains generally to the field of cancer therapy. More specifically, the invention pertains to the field of inhibiting tumor neovascularization, or angiogenesis. More specifically, the invention pertains to the field of blocking tumor blood flow through the induction of immune responses targeting tumor derived vascular channels, endothelial cells, or endothelial-like cells.

### BACKGROUND OF THE INVENTION

The establishment of a critical role of the angiogenic switch in tumorigenesis, tumor progression and tumor metastasis has made the rationale behind the development of anti-angiogenesis therapy clear. Unfortunately, the ability to attain long-term efficacy of therapy for all cancer-types, in order to reduce cancer to a dormant, chronic manageable disease without increasing morbidity from side effects, has not yet been achieved, as has been for diseases like HIV.

The VEGF pathway has been the first pathway of angiogenesis associated with tumors that was discovered, and sufficient rationale existed to support the utilization of antibodies to this pathway clinically. This cytokine is an endothelial cell-specific mitogen in vitro and an angiogenic inducer in a variety of in vivo models. The lack of oxygen to cells (hypoxia) has been shown to be a major inducer of VEGF gene transcription. The tyrosine kinases Flt-1(VEGFR-1) and Flk-1/KDR (VEGFR-2) are high-affinity VEGF receptors. The role of VEGF in developmental angiogenesis is emphasized by the finding that loss of a single VEGF allele results in defective vascularization and early embryonic lethality. VEGF is critical also for reproductive and bone angiogenesis. Substantial evidence also implicates VEGF as a mediator of pathological angiogenesis. In situ hybridization studies demonstrate expression of VEGF mRNA in the majority of human tumors [1]. Furthermore, elevated concentrations of VEGF not only in the tumor microenvironment, but also systemically have been detected in glioma [2, 3], and several other tumors [4].

Unfortunately, it is now well recognized that all three FDA-approved VEGF pathway inhibitors (anti-VEGF bevacizumab or Avastin, AntiVEGFR2 sunitinib, and sorafanib) result in only temporary improvements in the form of tumor growth blockade or shrinkage, and only for certain cancers despite most, if not all cancer types exhibiting pathological angiogenesis. Moreover, while anti-VEGF pathway therapies have reduced primary tumor growth and metastasis in preclinical studies, recent animal studies have reported that sunitinib and an anti-VEGFR2 antibody, DC101, increased metastasis of tumor cells despite inhibition of primary tumor growth and increased overall survival in some cases. Specifically, it has been demonstrated that these angiogenesis inhibitors targeting the VEGF pathway demonstrate antitumor effects in mouse models of pancreatic neuroendocrine carcinoma and glioblastoma but concomitantly elicit tumor adaptation and progression to stages of greater malignancy, with heightened invasiveness and in some cases increased lymphatic and distant metastasis. Increased invasiveness is also seen by genetic ablation of the Vegf-A gene in both models, substantiating the results of the pharmacological inhibitors [4-7].

Addressing this “antiangiogenesis therapy conundrum,” cumulative observations have suggested several mechanisms of evasive and intrinsic resistance [8] such as: a) activation and/or upregulation of alternative pro angiogenic pathways, b) recruitment of bone marrow-derived pro-angiogenic cells, c) increased pericyte coverage for the tumor vasculature, attenuating the need for VEGF signaling; d) activation and enhancement of invasion and metastasis to provide access to normal tissue vasculature without obligate neovascularization; [for intrinsic resistance]: e) pre-existing multiplicity of redundant pro-angiogenic signals; f) pre-existing inflammatory cell-mediated vascular protection; g) tumor hypovascularity; and h) invasive and metastatic co-option of normal vessels without requisite angiogenesis [9-12]. Accordingly, there is a need in the art for development of new approaches towards blockade of tumor angiogenesis. One approach is through the stimulation of immune attack against the blood vessels that are selective to the tumor. Below is a description of immunology associated with tumors to provide the practitioner of the invention with a background.

The human immune system may viewed in a very general sense as being divided into two arms, which are typically described or referred to as “innate immunity” and “adaptive immunity.”

The innate arm of the immune system is predominantly responsible for an initial inflammatory response via a number of soluble factors, including the complement system and the chemokine/cytokine system; and a number of specialized cell types including mast cells, macrophages, dendritic cells (DCs), and natural killer cells.

In contrast, the adaptive immune arm involves a delayed and a longer lasting antibody response together with CD8+ and CD4+ T cell responses that play a critical role in immunological memory against an antigen. A third arm of the immune system may be identified as involving Gamma delta T cells and T cells with limited T cell receptor repertoires such as NKT cells and MATT cells.

For an effective immune response to an antigen, with antigens being bacterial, viral, or as in the case of the invention, tumor derived, antigen presenting cells (APCs) must process and display the antigen in a proper MHC context to aT cell, which then will result in either T cell stimulation of cytotoxic and helper T cells. Following antigen presentation successful interaction of co-stimulatory molecules on both APCs and T cells must occur or activation will be aborted. GM-CSF and IL-12 serve as effective pro- inflammatory molecules in many tumor models. For example, GM-CSF induces myeloid precursor cells to proliferate and differentiate into dendritic cells (DCs) although additional signals are necessary to activate their maturation to effective antigen-presenting cells necessary for activation of T cells. Barriers to effective immune therapies include tolerance to the targeted antigen that can limit induction of cytotoxic CD8 T cells of appropriate magnitude and function, poor trafficking of the generated T cells to sites of malignant cells, and poor persistence of the induced T cell response.

DCs that phagocytose tumor-cell debris process the material for major histocompatibility complex (MHC) presentation, upregulate expression of costimulatory molecules, and migrate to regional lymph nodes to stimulate tumor-specific lymphocytes. This pathway results in the proliferation and activation of CD4+ and CD8+ T cells that react to tumor-associated antigens. Indeed, such cells can be detected frequently in the blood, lymphoid tissues, and malignant lesions of patients. New insights into the mechanisms underlying immune-evasion, together with combination treatment regimens that potentiate the potency of therapeutic vaccination—either directly or indirectly—through combination with immune checkpoint inhibitors or other therapies, have served as a basis for the development of vaccines that induce effective antitumor immunity.

### DETAILED DESCRIPTION OF THE INVENTION

The invention provides means of inducing immunity to endothelial cells that are derived from tumors themselves, as well as endothelial-like cells, and intratumoral vascular channels. In one embodiment, the invention provides means of generating such tumor-derived endothelial cells for utilization as a source of tumor antigens. Specifically, tumor cells are subjected to conditions resembling the tumor microenvironment and subsequently cells are isolated possessing endothelial markers. Said cells may be expanded by culture in vitro in endothelial culture media, or may be immortalized to develop a stable cellular source. Conditions associated with tumor cell differentiation into endothelial like cells, or cells of tumor or chimeric origin possessing ability to supple the tumor with blood.

In another embodiment of the invention primary tumor cells are selected from a tumor biopsy. Processes and procedures for isolation of primary tumor cells are known in the art [13].

It is known in the art that tumor cells spontaneously fuse with non-tumor cells [14], which has been associated with malignant progression. Additionally, various host cells are useful in assisting or accelerating tumor growth. Studies of adoptive T cell immunotherapy [15] along with recently reported positive clinical results in non-Hodgkins lymphoma [16, 17] and prostate cancer immunotherapy targeting tumor-associated antigens (TAAs) have provided proof of concept that the immune system can support a clinically effective anti-tumor immune response. Although the benefits of anti-tumor immunotherapy has not been demonstrated in a wide range of tumor types, it has been postulated that the missing critical element is a sufficiently potent, readily translatable cancer vaccine strategy [18]. Patients with cancer can have endogenous or immunotherapy elicited humoral and cellular responses to several tumor-associated antigens (TAAs) [19-28], however, these have generally been of sub-optimal magnitude with elusive clinical efficacy. Additionally, breast cancer patients with significant inflammatory infiltrates, i.e. medullary breast carcinoma, have significantly improved survival despite greater cellular anaplasia [29-31]. Thus in one embodiment of the invention immunity is generated towards cells associated providing blood supply to tumor. While in our previous patent application we addressed the issue of host-derived endothelial cells, in this patent we disclose the generation of immunity towards tumor-derived blood vessels. There are several TAAs that have been identified in cancer consisting of overexpressed normal proteins and mutated proteins that are normally found in breast tissue, however, only a minority of the TAAs that have been discovered so far are immunogenic, which limits the potential use for immunotherapy. In addition, while the overwhelming majority of TAAs are expressed in tumor cells, they are typically also expressed in a variety of normal cells, e.g. the breast cancer TAAs; epidermal growth factor receptors (HER2), carcinoembryonic antigen (CEA), mucin (MUCI), the tumor suppressor protein p53, and telomerase reverse transcriptase (TERT). Thus, they are recognized by the immune system as self-molecules, and the immune system has protective mechanisms for preventing recognition of self-tissue antigens and autoimmune responses. Additionally, tumors employ other mechanisms for escaping immune surveillance, such as: (i) low level expression of MHC class I molecules [32]; (ii) lack of expression of B7 (CD80/CD86) co-stimulatory molecules [33]; (iii) production of cytokines that stimulate the accumulation of immune-suppressor cells [34, 35]; and (iv) ineffective processing and presentation of self-antigens by “professional” antigen-presenting cells (APC) [36]. This probably explains why TAA or tumor cell vaccines that have been used in clinical trials generally do not induce strong protective immunity [15]. Identification of novel TAA that are not expressed on normal cells may provide an attractive alternative particularly if combined with potent immunotherapeutic platforms, because these antigens are less likely to be subject to the tolerogenic mechanisms that limit immune responses to “self” antigens and therefore, may be better immunogens. Accordingly the invention seeks to induce an immunity towards suppressive elements associated with the tumor, through blocking said suppressive elements by vaccination against various tumor components associated with the tumor microenvironment.

To facilitate understanding and practice of the invention, a list of definitions is provided below.

By “a cell” we refer to one or a plurality of cells and refers to all types of cells including hematopoietic and cancer cells.

By “stem cell” we refer to a cell that has the ability for self-renewal. Non-cancerous stem cells have the ability to differentiate where they can give rise to specialized cells.

By “effective amount” we refer to a means a quantity sufficient to, when administered to an animal, effect beneficial or desired results, including clinical results, and as such, an “effective amount” depends upon the context in which it is being applied.

By “oligonucleotide” we refer to unmodified DNA or RNA or modified DNA or RNA. For example, the nucleic acid molecules or polynucleotides of the disclosure can be composed of single- and double stranded DNA, DNA that is a mixture of single- and double-stranded regions, single- and double- stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple- stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

By “animal” we refer to all members of the animal kingdom, preferably mammal. The term “mammal” as used herein is meant to encompass, without limitation, humans, domestic animals such as dogs, cats, horses, cattle, swine, sheep, goats, and the like, as well as wild animals. In an embodiment, the mammal is human.

By “interfering RNA” or “RNAi” or “interfering RNA sequence,” we refer to double-stranded RNA (i.e., duplex RNA) that targets (i.e., silences, reduces, or inhibits) expression of a target gene (Le., by mediating the degradation of mRNAs which are complementary to the sequence of the interfering RNA) when the interfering RNA is in the same cell as the target gene. Interfering RNA thus refers to the double stranded RNA formed by two complementary strands or by a single, self-complementary strand. Interfering RNA typically has substantial or complete identity to the target gene. The sequence of the interfering RNA can correspond to the full length target gene, or a subsequence thereof. Interfering RNA includes small-interfering “RNA” or “siRNA,” i.e., interfering RNA of about 15-60, 15-50, 15-50, or 15-40 (duplex) nucleotides in length, more typically about, 15-30, 15-25 or 19-25 (duplex) nucleotides in length, and is preferably about 20-24 or about 21-22 or 21-23 (duplex) nucleotides in length (e.g., each complementary sequence of the double stranded siRNA is 15-60, 15-50, 15-50, 15-40, 15-30, 15-25 or 19-25 nucleotides in length, preferably about 20-24 or about 21-22 or 21-23 nucleotides in length, and the double stranded siRNA is about 15-60, 15-50, 15-50, 15-40, 15-30, 15-25 or 19-25 preferably about 20-24 or about 21-22 or 21-23 base pairs in length). siRNA duplexes may comprise 3′ overhangs of about 1 to about 4 nucleotides, preferably of about 2 to about 3 nucleotides and 5′ phosphate termini, The siRNA can be chemically synthesized or maybe encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., PNAS USA 99: 9942-7 (2002); Calegari et al., PNAS USA 99: 14236 (2002); Byrom et al., Ambion TechNotes 10(1): 4-6 (2003); Kawasaki et al.; Nucleic Acids Res. 31:981-7 (2003); Knight and Bass, Science 2.93: 2269-71 (2001); and Robertson et al., J. Biol. Chem. 243: 82 (1968)). Preferably, dsRNA are at least 50 nucleotides to about 100, 200, 300, 400 or 500 nucleotides in length. A dsRNA may be as long as 1000, 1500, 2000, 5000 nucleotides in length or longer. The dsRNA can encode for an entire gene transcript or a partial gene transcript.

By “siRNA” we refer to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules or shRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

By “antisense nucleic acid” as used herein means a nucleotide sequence that is complementary to its target e.g. a tumor derived immune suppressive transcription product such as IL10. The nucleic acid can comprise DNA, RNA or a chemical analog, that binds to the messenger RNA produced by the target gene. Binding of the antisense nucleic acid prevents translation and thereby inhibits or reduces target protein expression. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

By “treat” we mean to ameliorate at least one symptom of the disorder. In some embodiments, a treatment can result in a reduction in tumor size or number, or a reduction in tumor growth or growth rate.

By cellular proliferative and/or differentiative disorders we refer to cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and origin.

By “cancer”, “hyperproliferative” and “neoplastic” refer to cells having the capacity for autonomous growth, i,e., an abnormal state or condition characterized by rapidly proliferating cell growth. Hyperproliferative and neoptastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasms” include malignancies of the various organ systems, e.g., affecting the nervous system, lung, breast, thyroid, lymphoid, gastrointestinal, and genito-urinary tract, as well as adenocarcinomas, which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

By “chromatin mass” is meant more than one chromosome not enclosed by a membrane. Preferably, the chromatin mass contains all of the chromosomes of a cell. A chromatin mass containing condensed chromosomes may be formed by exposure of a nucleus to a mitotic reprogramming media (e.g., a mitotic extract), or a chromatin mass may be isolated from mitotic cells as described herein. Alternatively, a chromatin mass containing decondensed or partially condensed chromosomes may be generated by exposure of a nucleus to one of the following, as described herein: a mitotic reprogramming media (e.g., a mitotic extract) in the presence of an anti-NuMA antibody, a detergent and salt solution, or a protein kinase solution. A chromatin mass may be formed naturally or artificially induced. An exemplary naturally-occurring chromatin mass includes a set of metaphase chromosomes, which are partially or maximally condensed chromosomes that are not surrounded by a membrane and that are found in, or isolated from, a mitotic cell. Preferably, the metaphase chromosomes are discrete chromosomes that are not physically touching each other. Exemplary artificially induced chromatin masses are formed from exposure to a reprogramming media, such as a solution containing factors that promote chromosome condensation, a mitotic extract, a detergent and salt solution, or a protein kinase solution. Artificially induced chromatin masses may contain discrete chromosomes that are not physically touching each other or may contain two or more chromosomes that are in physical contact. If desired, the level of chromosome condensation may be determined using standard methods by measuring the intensity of staining with the DNA stain, DAPI. As chromosomes condense, this staining intensity increases. Thus, the staining intensity of the chromosomes may be compared to the staining intensity for decondensed chromosomes in interphase (designated 0% condensed) and maximally condensed chromosomes in mitosis (designated 100% condensed). Based on this comparison, the percent of maximal condensation may be determined. Preferred condensed chromatin masses are at least 50, 60, 70, 80, 90, or 100% condensed. Preferred decondensed or partially condensed chromatin masses are less than 50, 40, 30, 20, or 10% condensed.

By “nucleus” is meant a membrane-bounded organelle containing most or all of the DNA of a cell. The DNA is packaged into chromosomes in a decondensed form. Preferably, the membrane encapsulating the DNA includes one or two lipid bilayers or has nucleoporins.

By “donor cell” is meant a cell from which a nucleus or chromatin mass is derived.

By “cytoplast” is meant a membrane-enclosed cytoplasm. Preferably, the cytoplast does not contain a nucleus, chromatin mass, or chromosome. Cytoplasts may be formed using standard procedures. For example, cytoplasts may be derived from nucleated or enucleated cells. Alternatively, cytoplasts may be generated using methods that do not require an intact cell to be used as the source of the cytoplasm or as the source of the membrane. In one such method, cytoplasts are produced by the formation of a membrane in the presence of cytoplasm under conditions that allow encapsulation of the cytoplasm by the membrane.

By “permeabilization” is meant the formation of pores in the plasma membrane or the partial or complete removal of the plasma membrane.

By “reprogramming media” is meant a solution that allows the removal of a factor from a nucleus, chromatin mass, or chromosome or the addition of a factor from the solution to the nucleus, chromatin mass, or chromosome. Preferably, the addition or removal of a factor increases or decreases the level of expression of an mRNA or protein in the donor cell, chromatin mass, or nucleus or in a cell containing the reprogrammed chromatin mass or nucleus. In another embodiment, incubating a permeabilized cell, chromatin mass, or nucleus in the reprogramming media alters a phenotype of the permeabilized cell or a cell containing the reprogrammed chromatin mass or nucleus relative to the phenotype of the donor cell. In yet another embodiment, incubating a permeabilized cell, chromatin mass, or nucleus in the reprogramming media causes the permeabilized cell or a cell containing the reprogrammed chromatin mass or nucleus to gain or loss an activity relative to the donor cell. Exemplary reprogramming medias include solutions, such as buffers, that do not contain biological molecules such as proteins or nucleic acids. Such solutions are useful for the removal of one or more factors from a nucleus, chromatin mass, or chromosome. Other preferred reprogramming medias are extracts, such as cellular extracts from cell nuclei, cell cytoplasm, or a combination thereof. Yet other reprogramming medias are solutions or extracts to which one or more naturally-occurring or recombinant factors (e.g., nucleic acids or proteins such as DNA methyltransferases, histone deacetylases, histones, nuclear lamins, transcription factors, activators, repressors, growth factors, hormones, or cytokines) have been added, or extracts from which one or more factors have been removed. Still other reprogramming medias include detergent and salt solutions and protein kinase solutions. In some embodiments, the reprogramming media contains an anti-NuMA antibody. By “interphase reprogramming media” is meant a media (e.g., an interphase cell extract) that induces chromatin decondensation and nuclear envelope formation. By “mitotic reprogramming media” is meant a media (e.g., a mitotic cell extract) that induces chromatin condensation and nuclear envelope breakdown. If desired, multiple reprogramming media may be used simultaneously or sequentially to reprogram a donor cell, nucleus, or chromatin mass.

By “addition of a factor” is meant the binding of a factor to chromatin, a chromosome, or a component of the nuclear envelope, such as the nuclear membrane or nuclear matrix. Alternatively, the factor is imported into the nucleus so that it is bounded or encapsulated by the nuclear envelope. Preferably, the amount of factor that is bound to a chromosome or located in the nucleus increases by at least 25, 50, 75, 100, 200, or 500%.

By “removal of factor” is meant the dissociation of a factor from chromatin, a chromosome, or a component of the nuclear envelope, such as the nuclear membrane or nuclear matrix. Alternatively, the factor is exported out of the nucleus so that it is no longer bounded or encapsulated by the nuclear envelope. Preferably, the amount of factor that is bound to a chromosome or located in the nucleus decreases by at least 25, 50, 75, 100, 200, or 500%.

By “enrichment or depletion of a factor” is meant the addition or removal of a naturally-occurring or recombinant factor by at least 20, 40, 60, 80, or 100% of the amount of the factor originally present in the reprogramming media. Alternatively, a naturally-occurring or recombinant factor that is not naturally present in the reprogramming media may be added. Preferred factors include proteins such as DNA methyltransferases, histone deacetylases, histones, nuclear lamins, transcription factors, activators, repressors, growth factors, cytokines, and hormones; membrane vesicles; and organelles. In one preferred embodiment, the factor is purified prior to being added to the reprogramming media, as described below. Alternatively, one of the purification methods described below may be used to remove an undesired factor from the reprogramming media.

By “purified” is meant separated from other components that naturally accompany it. Typically, a factor is substantially pure when it is at least 50%, by weight, free from proteins, antibodies, and naturally- occurring organic molecules with which it is naturally associated. Preferably, the factor is at least 75%, more preferably, at least 90%, and most preferably, at least 99%, by weight, pure. A substantially pure factor may be obtained by chemical synthesis, separation of the factor from natural sources, or production of the factor in a recombinant host cell that does not naturally produce the factor. Proteins, vesicles, chromosomes, nuclei, and other organelles may be purified by one skilled in the art using standard techniques such as those described by Ausubel et al. (Current Protocols in Molecular Biology, John Wiley & Sons, New York, 2000). The factor is preferably at least 2, 5, or 10 times as pure as the starting material, as measured using polyacrylamide gel electrophoresis, column chromatography, optical density, HPLC analysis, or western analysis (Ausubel et al., supra). Preferred methods of purification include immunoprecipitation, column chromatography such as immunoaffinity chromatography, magnetic bead immunoaffinity purification, and panning with a plate-bound antibody.

By “mRNA or protein specific for one cell type” is meant an mRNA or protein that is expressed in one cell type at a level that is at least 10, 20, 50, 75, or 100 fold greater than the expression level in all other cell types. Preferably, the mRNA or protein is only expressed in one cell type.

By “mutation” is meant an alteration in a naturally-occurring or reference nucleic acid sequence, such as an insertion, deletion, frameshift mutation, silent mutation, nonsense mutation, or missense mutation. Preferably, the amino acid sequence encoded by the nucleic acid sequence has at least one amino acid alteration from a naturally-occurring sequence. Examples of recombinant DNA techniques for altering the genomic sequence of a cell, embryo, fetus, or mammal include inserting a DNA sequence from another organism (e.g., a human) into the genome, deleting one or more DNA sequences, and introducing one or more base mutations (e.g., site-directed or random mutations) into a target DNA sequence. Examples of methods for producing these modifications include retroviral insertion, artificial chromosome techniques, gene insertion, random insertion with tissue specific promoters, homologous recombination, gene targeting, transposable elements, and any other method for introducing foreign DNA. All of these techniques are well known to those skilled in the art of molecular biology (see, for example, Ausubel et al., supra). Chromatin masses, chromosomes, and nuclei from transgenic cells, tissues, organs, or mammals containing modified DNA may be used in the methods of the invention.

By “substantially identical” is meant having a sequence that is at least 60, 70, 80, 90, or 100% identical to that of another sequence or to a naturally-occurring sequence. Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wisc. 53705). This software program matches similar sequences by assigning degrees of homology to various substitutions, deletions, and other modifications.

By “immortilized” is meant capable of undergoing at least 25, 50, 75, 90, or 95% more cell divisions than a naturally-occurring control cell of the same cell type, genus, and species as the immortalized cell or than the donor cell from which the immortalized cell was derived. Preferably, an immortalized cell is capable of undergoing at least 2, 5, 10, or 20-fold more cell divisions than the control cell. More preferably, the immortalized cell is capable of undergoing an unlimited number of cell divisions. Examples of immortalized cells include cells that naturally acquire a mutation in vivo or in vitro that alters their normal growth-regulating process. Other preferred immortalized cells include hybridoma cells which are generated using standard techniques for fusion of a myeloma with a B-cell (Mocikat, J. Immunol. Methods 225:185-189, 1999; Janak et al., Hum. Antibodies Hybridomas 3:177-185, 1992; Srikumaran et al., Science 220:522, 1983). Still other preferred immortalized cells include cells that have been genetically modified to express an oncogene, such as ras, myc, ab1, bc12, or neu, or that have been infected with a transforming DNA or RNA virus, such as Epstein Barr virus or SV40 virus (Kumar et al., Immunol. Lett. 65:153-159, 1999; Knight et al., Proc. Nat. Acad. Sci. USA 85:3130-3134, 1988; Shammah et al., J. Immunol. Methods 160-19-25, 1993; Gustafsson and Hinkula, Hum. Antibodies Hybridomas 5:98-104, 1994; Kataoka et al., Differentiation 62:201-211, 1997; Chatelut et al., Scand. J. Immunol. 48:659-666, 1998). Cells can also be genetically modified to express the telomerase gene (Rogues et al., Cancer Res. 61:8405-8507, 2001).

In one embodiment, the invention teaches culture of tumor cells in hypoxic conditions, together with agents capable of stimulating endothelial, endothelial-like or tumor vascular channel cells. Once these cells are generated, they are assayed for blood vessel generating potential, and utilized as antigenic sources either alone, or through use as antigens for pulsing antigen presenting cells such as B cells or in a preferred embodiment, dendritic cells.

When investigators assessed efficacy of anti-VEGF (Avastin) and anti-VEGF receptor (sunitinib) in an orthotopic glioma model, it was found that VEGF receptor inhibition was associated with induction of upregulated tumor hypoxia, which stimulated infiltration of macrophages into the tumor. At the time of tumor progression after therapy, a significant increase in CD11b(+)/Gr1(+) granulocyte infiltration was observed, and tumors developed aggressive mesenchymal features and increased stem cell marker expression [37]. Accordingly, in one embodiment of the invention, the use of vaccination against tumor derived endothelial cells, endothelial-like cells, or intratumor vascular channels is performed in conjunction with approaches that reduce myeloid suppressor cells such as CD11b(+)/Gr1(+) granulocyte.

In one embodiment of the invention suppression of VEGF is performed to reduce concentrations of myeloid suppressor cells through placing the patient on a caloric restriction diet [38]. In one embodiment of the invention suppression of VEGF is performed to reduce concentrations of myeloid suppressor cells through administration of the FDA approved drug dapsone [39]. Means of generating antibodies of human origin are well known in the art, with example U.S. Pat. Nos. 5,567,610 and 5,229,275 being instructive. Such antibodies can be used for selectively blocking molecules associated with tumor progression such as VEGF and other soluble mediators.

In one embodiment the present invention is to provide methods for altering the characteristics or functions of tumor cells in vitro to simulate in vivo tumor conditions and to extract from said conditions tumor cells differentiating into endothelial or endothelial like cells, or vascular channels. In particular, these methods involve incubating a nucleus or chromatin mass from a donor cell, specifically the cancer for which cancer endothelial cells are sought to be generated, with a reprogramming media (e.g., a cell extract) under conditions that allow nuclear or cytoplasmic components such as transcription factors to be added to, or removed from, the nucleus or chromatin mass. The cells providing the programming media are endothelial cells, or other cells that would be found in the cancer microenvironment such as monocytes, neutrophils, basophils or mast cells. Preferably, the added transcription factors promote the expression of mRNA or protein molecules found in cells of the desired cell type, and the removal of transcription factors that would otherwise promote expression of mRNA or protein molecules found in the donor cell, thus altering the phenotype towards a tumor endothelial or endothelial like or vascular channel of tumor cell. If desired, the chromatin mass may then be incubated in an interphase reprogramming media (e.g., an interphase cell extract) to reform a nucleus that incorporates desired factors from either reprogramming media. Then, the nucleus or chromatin mass is inserted into a recipient cell or cytoplast, forming a reprogrammed cell of the desired cell type. In a related method, a permeabilized cell is incubated with a reprogramming media (for example cell extract) to allow the addition or removal of factors from the cell, and then the plasma membrane of the permeabilized cell is resealed to enclose the desired factors and restore the membrane integrity of the cell. If desired, the steps of any of these methods may be repeated one or more times or different reprogramming methods may be performed sequentially to increase the extent of reprogramming, resulting in a greater alteration of the mRNA and protein expression profile in the reprogrammed cell. Furthermore, reprogramming medias may be made representing combinations of cell functions (e.g., medias containing extracts or factors from multiple cell types) to produce unique reprogrammed cells possessing characteristics of multiple cell types. In a specific embodiment the reprogramming media may contain type 2 monocytes, or extracts thereof, or exosomes or microvesicular particles derived from said cells.

In one embodiment of the invention, ValloVax is utilized to induce immunity against tumor generated vasculature. ValloVax is prepared from full term placentas that are collected from delivery room under informed consent. Fetal membranes are manually peeled back and the villous tissue is isolated from the placental structure. Villous tissue is subsequently washed with cold saline to remove blood and scissors are used to mechanically digest the tissue, 25 grams of minced tissue is incubated with approximately 50 ml of HBSS with 25 mM of HEPES and 0.28% collagenase, 0.25% dispase, and 0.01% DNAse at 37 Celsius. The mixture of minced placental villus tissue and digesting solution is incubated under stirring conditions for three incubation periods of 20 minutes each. Ten minutes after the first incubation period and immediately after the second and third incubation periods, the DNAse is added to make up a total concentration of DNase, by volume, of 0.01%; In the first and second incubations, the incubation flask is set at an angle, and the tissue fragments are allowed to settle for approximately 1 minute, with 35 ml of the supernantant cell suspension being collected and replaced by 38 ml (after the first digestion) or 28 ml (after the second digestion) of fresh digestion solution. After the third digestion the

Whole supernatant is collected; The supernatant collected from all three incubations is pooled and is poured through approximately four layers of sterile gauze and through one layer of 70 micro meter polyester mesh. The filtered solution is then centrifuged for 1000 g for 10 minutes through diluted new born calf serum, said new born calf serum diluted at a ratio of 1 volume saline to 7 volumes of new born calf serum; The pooled pellet is then resuspended in 35 ml of warm DMEM with 25 mM HEPES containing 5 mg DNase I; The suspension is then mixed with 10 ml of 90% Percoll to give a final density of 1.027 g/ml and is centrifuged at 550 g for 10 minutes with the centrifuge brake off; The pellet is then washed in HBSS and cells are incubated for 48 hours in complete DMEM media containing 100 IU of IFN-gamma per mi. Subsequent to incubation cells were either used: a) unmanipulated; b) used as a lysate, with 10 freeze thaw cycles in liquid nitrogen, subsequent to which cells were filtered through a 0.2 micron filter; c) mitotically inactivated by irradiation at 10 Gy.

In another embodiment, cells of ValloVax are fused with tumor cells to provide an optimal tumor vaccine.

In one embodiment, the invention provides a method of reprogramming a cancer cell in vitro to resemble in vivo cancer cells. This method involves incubating a nucleus from the cancer cell with a reprogramming media (e.g., a cell extract, such as monocytic, endothelial or type 2 monocytic) under conditions that allow the removal of a factor from the nucleus or the addition of a factor to the nucleus. Then the nucleus or a chromatin mass formed from incubation of the nucleus in the reprogramming media is inserted into a recipient cell or cytoplast, thereby forming a reprogrammed cell. In one preferred embodiment, the nucleus is incubated with an interphase reprogramming media (e.g., an interphase cell extract). Further, the invention provides another method of reprogramming a cell. This method involves incubating a chromatin mass with a reprogramming media (e.g., a cell extract) under conditions that allow the removal of a factor from the chromatin mass or the addition of a factor to the chromatin mass. Then the chromatin mass or nucleus formed from incubation of the chromatin mass in a reprogramming media (e.g., an interphase extract) is inserted into a recipient cell or cytoplast, thereby forming a reprogrammed cell. In one preferred embodiment, the chromatin mass is generated by incubating a nucleus from a donor cell in a detergent and salt solution, in a protein kinase solution, or in a mitotic reprogramming media in the presence or absence of an antibody to NuMA or to another protein of the nucleus. In another preferred embodiment, the chromatin mass is isolated from mitotic cells. In another related aspect, the invention provides yet another method of reprogramming a cell.

This method involves incubating a permeabilized cell with a reprogramming media (e.g., a cell extract) under conditions that allow the removal of a factor from the nucleus or chromatin mass of the permeabilized cell or the addition of a factor to the nucleus or chromatin mass, thereby forming a reprogrammed cell. In one preferred embodiment, the permeabilized cell is incubated with an interphase reprogramming media (e.g., an interphase cell extract). Preferably, the nucleus in the permeabilized cell remains membrane-bounded, and the chromosomes in the nucleus do not condense during incubation with this interphase reprogramming media. In another preferred embodiment, a chromatin mass is formed from incubation of the permeabilized cell in a mitotic reprogramming media. In yet another preferred embodiment, the reprogrammed cell is incubated under conditions that allow the membrane of the reprogrammed cell to reseal. If desired, the permeabilized cell may be formed by incubating an intact cell with a detergent, such as digitonin, or a bacterial toxin, such as Streptolysin O. Generated endothelial-like cells or vascular channel cells may be fused with dendritic cells or in a preferred embodiment with immature dendritic cells for the stimulation of immunity. Methods for stimulation of fusion with dendritic cells is known in the art and is described in the following references [40, 41]. In some embodiments the fused endothelial-like cells or vascular channel cells together with dendritic cells are treated with an activation stimuli to further increase immunogenicity. Immunogenicity may be assessed by expression of markers associated with costimulation of T cells such as CD80 and CD86. Additional methods of assessing immunogenicity include mixed lymphocyte reaction as assessed by responding cell proliferation or production of Th1cytokines. The maturation stimuli of the fused cells may be activation of the CD40-CD40L pathway which is described in detail in the following reference [42].

The invention also provides reprogrammed cells generated, using any method of the invention or a combination of methods of the invention. These cells, chimeric, or altered cells are useful as a source of antigen for immunization of patients with the desire to successfully destroy tumor vasculature. In many cases the tumor vasculature appears to be derived from the tumor itself and not from endothelial precursor cells. This provides an element of genetic instability of the tumor vasculature, and also results in decreased dependence on VEGF, which may explain why in many cases VEGF provides only a temporary solution to cancer. Through stimulation of immunity by immunization, not only are therapeutic means disclosed, but the discovery of novel antigens that are specific to tumor vasculature is contemplated within the present invention through the utilization of techniques well known in the art such as SEREX, or other means of identifying antigens that are bound to antibodies generated during an immune response, such as phage display, which are described in the following references [43-54].

In one embodiment of the invention, the invention teaches a cell that expresses a combination of two or more endogenous mRNA molecules or endogenous proteins that is not expressed by the tumor cell in vitro but by a tumor cell in vivo. In a related aspect, the invention features a cell that expresses a combination of two or more endogenous mRNA molecules or endogenous proteins at a level that is at least 10, 20, 50, 75, or 100 fold greater than the expression level of the corresponding mRNA molecules or proteins in any naturally-occurring cell. Methods of cytoplasmic transfer utilized for reprogramming of cells have been previously published with other cell systems and are incorporated by reference [55, 56]. In preferred embodiments of the above aspects, the cell expresses a combination of 5, 10, 25, 50, 75, 100, 150, 300, or more endogenous mRNA molecules or endogenous proteins that is not expressed by a naturally-occurring cell. In another preferred embodiment, the cell expresses 1, 3, 5, 10, 25, 50, 100, or more endogenous mRNA molecules or endogenous proteins that are specific for one cell type and expresses 1, 3, 5, 10, 25, 50, 100, or more endogenous mRNA molecules or endogenous proteins that are specific for another cell type. In other preferred embodiments, the cell has a combination of 2, 5, 10, 25, 50, 75, 100, 150, 300, or more activities or phenotypes that are not exhibited in a naturally-occurring. In yet other preferred embodiments, the cell is able to divide or is immortalized and expresses tenascin C other protein expressed by tumor vascular cells. In yet another embodiment, the cell is formed from the reprogramming of a donor cancer cell, nucleus, or chromatin mass, and the reprogrammed cell expresses one or more endothelial cell markers such as CD31 or CD34 at a level that is at least 25, 50, 75, 90, or 95% higher that the corresponding level in the donor under the same conditions. These methods for reprogramming cells are useful for the generation of cells of a desired cell type, for example, for medical applications. Accordingly, the invention also provides methods for the treatment or prevention of disease in a mammal that include administering a reprogrammed cell to the mammal.

In one embodiment the invention provides another method of generating a tumor cell possessing endothelial characteristics by incubating a permeabilized cancer cell with a reprogramming media (e.g., a cell extract) under conditions that allow the removal of a factor from the nucleus or chromatin mass of the permeabilized cell or the addition of a factor to the nucleus or chromatin mass. The reprogrammed cell formed from this step is administered together with an immunological adjuvant to induce immunity to tumor vasculature. In one preferred embodiment, the permeabilized cell is incubated with an interphase reprogramming media. Preferably, the nucleus in the permeabilized cell remains membrane-bounded, and the chromosomes in the nucleus do not condense during incubation with the interphase reprogramming media. In another preferred embodiment, a chromatin mass is formed from incubation of the permeabilized cell in a mitotic reprogramming media. In yet another preferred embodiment, the reprogrammed cell is incubated under conditions that allow the membrane of the reprogrammed cell to reseal prior to being administered to the mammal. Preferably, the permeabilized cell is from the mammal in need of that cell type. In another preferred embodiment, the permeabilized cell is formed by incubating an intact cell with a detergent, such as digitonin, or a bacterial toxin, such as Streptolysin O.

In preferred embodiments of various aspects of the invention, at least 1, 5, 10, 15, 20, 25, 50, 75, 100, 150, 200, 300, or more mRNA or protein molecules are expressed in the reprogrammed cell that are not expressed in the donor or permeabilized cell. In another preferred embodiment, the number of mRNA or protein molecules that are expressed in the reprogrammed cell, but not expressed in the donor or permeabilized cell, is between 1 and 5, 5 and 10, 10 and 25, 25 and 50, 50 and 75, 75 and 100, 100 and 150, 150 and 200, or 200 and 300, inclusive. Preferably, at least 1, 5, 10, 15, 20, 25, 50, 75, 100, 150, 200, 300, or more mRNA or protein molecules are expressed in the donor or permeabilized cell that are not expressed in the reprogrammed cell. In yet another preferred embodiment, the number of mRNA or protein molecules that are expressed in the donor or permeabilized cell, but not expressed in the reprogrammed cell, is between 1 and 5, 5 and 10, 10 and 25, 25 and 50, 50 and 75, 75 and 100, 100 and 150, 150 and 200, or 200 and 300, inclusive. Preferably, the mRNA or protein molecules are specific for the cell type of the donor, permeabilized, or reprogrammed cell, such that the molecules are only expressed in cells of that particular cell type. In still another preferred embodiment, these mRNA or protein molecules are expressed in both the donor cell (i.e., the donor or permeabilized starting cell) and the reprogrammed cell, but the expression levels in these cells differ by at least 2, 5, 10, or 20-fold, as measured using standard assays (see, for example, Ausubel et al., Current Protocols in Molecular Biology, John Wiley & Sons, New York, 2000). In one embodiment, the size of the donor or permeabilized cell differs from that of the reprogrammed cell by at least 10, 20, 30, 50, 75, or 100%, as measured using standard methods. In another preferred embodiment, the volume of cytoplasm in the donor or permeabilized cell differs from that in the reprogrammed cell by at least 10, 20, 30, 50, 75, or 100%, based on standard methods. In yet another preferred embodiment, the reprogrammed cell has gained or lost an activity relative to the donor or permeabilized cell, such as production of matrix metalloproteases, angiogenic activity or chemotactic activity. In still other preferred embodiments, the reprogramming media is an interphase reprogramming media, such as an extract formed from cells synchronized in one or more of the phases of the cell cycle. In another embodiment, the reprogramming media is an extract formed from cells synchronized in mitosis or from unsynchronized cells. The reprogramming media is an extract from the cell type one wishes the donor or permeabilized cell to become, or the reprogramming media is a solution containing factors specific for the cell type one wishes the donor or permeabilized cell to become.

In one embodiment, the reprogramming media is modified by the enrichment or depletion of a factor, such as a DNA methyltransferase, histone deacetylase, histone, nuclear lamin, transcription factor, activator, repressor, growth factor, hormone, or cytokine. The reprogramming media may or may not contain exogenous nucleotides. In other embodiments, a chromatin mass in a reprogramming media or formed in a permeabilized cell is contacted with a vector having a nucleic acid encoding a gene of interest under conditions that allow homologous recombination between the nucleic acid in the vector and the corresponding nucleic acid in the genome of the chromatin mass, resulting in the alteration of the genome of the chromatin mass. Due to the lack of an intact plasma membrane and the lack of a nuclear membrane, a chromatin mass in a permeabilized cell may be easier to genetically modify than a naturally-occurring cell. Preferably, the chromatin mass or nucleus is purified from the reprogramming media prior to insertion into the recipient cell or cytoplast, or the reprogrammed cell is purified prior to administration into the mammal. Preferably, the donor or permeabilized cell is haploid (DNA content of n), diploid (2n), or tetraploid (4n), and the recipient cell is hypodiploid (DNA content of less than 2n), haploid, or enucleated.

In one embodiment, the invention describes the generation of a polyvalent antigenic source useful for the treatment of neuroblastoma. It is known that associated endothelial microvessels are lined by tumor-derived endothelial cells, that are genetically unstable and chemoresistant. In some situations the tumor derived endothelial cells account for the vast majority (about 80%) of the tumor surface area in touch with the blood [57]. The dedifferentiation associated transcription factor Oct-4 appears to be a marker useful for identification of progenitor cells within the tumor that give rise to tumor endothelial cells. Oct-4(+) cells are known to display a perivascular distribution, with 5% of them homing in perinecrotic areas. Oct-4(+) cells are known to be tumor-derived since they shared amplification of MYCN oncogene with malignant cells. Perivascular Oct-4(+) cells express stem cell-related, neural progenitor-related and NB-related markers, including surface Tenascin C.

In one embodiment, the in vitro generated tumor derived vascular cells are inactivated by radiation or mitotic toxins and are themselves modified to express and secrete one or more cytokines which stimulate induction, recruitment, and/or maturation of antigen presenting cells, preferably dendritic cells. One way of modifying cells to augment this activity is through transfection with GM-CSF. Thus, by way of example, the vascular cells may express a transgene encoding GM-CSF as described in U.S. Pat. Nos. 5,637,483, 5,904,920, 6,277,368 and 6,350,445, as well as in US Patent Publication No. 20100150946, each of which is expressly incorporated by reference herein. A form of GM-CSF- expressing genetically modified cancer cells or a “cytokine-expressing cellular vaccine” for the treatment of pancreatic cancer is described in U.S. Pat. Nos. 6,033,674 and 5,985,290, both of which are expressly incorporated by reference herein. Cytokines which may be expressed by such inactivated tumor vascular cells and/or bystander cells instead of, or together with, GM-CSF include, but are not limited to, one or more of CD40 ligand, IL-12, CCL3, CCL20, and CCL21. This list is not meant to be limiting. While it is preferred that the inactivated tumor vascular cells administered to the subject express one or more cytokines of interest, the tumor cell vascular line may be accompanied by an inactivated bystander cell line which expresses and secretes one or more cytokines which stimulate dendritic cell induction, recruitment, and/or maturation. The bystander cell line may provide all of the cytokines which stimulate dendritic cell induction, recruitment, and/or maturation, or may supplement cytokines which stimulate dendritic cell induction, recruitment, and/or maturation expressed and secreted by the inactivated tumor vascular cells. By way of example, immunomodulatory cytokine-expressing bystander cell lines are disclosed in U.S. Pat. Nos. 6,464,973, and 8,012,469, Dessureault et al., Ann. Surg. Oneal. 14: 869-84,2007, and Eager and Nemunaitis, Mol. Ther. 12: 18-27, 2005, each of which is expressly incorporated by reference herein.

It is known that other cancer agents may be utilized in conjunction with the immunotherapeutic targeting of tumor vasculature. When these agents are used care should be taken to select agents that do not comprise immunity. Numerous immunological assays exist including assessment of interferon gamma and TCR zeta chain activity. Agents useful in the art that have demonstrated cancer control or killing potential include: Such chemotherapeutic agents are known in the art and include but are not limited to: methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, doxorubicin, epirubicin, 5-fluorouracil, taxanes such as docetaxel and paclitaxel, leucovorin, levamisole, irinotecan, estramustine, etoposide, nitrosoureas such as carmustine and lomustine, vinca alkaloids, platinum compounds, mitomycin, gemcitabine, hexamethylmelamine, topotecan, tyrosine kinase inhibitors, tyrphostins, STI-571 or Gleevec.™. (imatinib mesylate), herbimycin A, genistein, erbstatin, and lavendustin A. taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, doxorubicin, epirubicin, 5-fluorouracil, taxanes such as docetaxel and paclitaxel, leucovorin, levamisole, irinotecan, estramustine, etoposide, nitrosoureas such as carmustine and lomustine, vinca alkaloids, platinum compounds, mitomycin, gemcitabine, hexamethylmelamine, topotecan, tyrosine kinase inhibitors, tyrphostinsherbimycin A, genistein, erbstatin, and lavendustin ABCNU, irinotecan, camptothecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a preferred embodiment, the anti-cancer agent can be, but is not limited to, a drug listed: Alkylating agents Nitrogen mustards: Cyclophosphamide Ifosfamide Trofosfamide Chlorambucil Nitrosoureas: Carmustine (BCNU) Lomustine (CCNU) Alkylsulphonates: Busulfan Treosulfan Triazenes: Dacarbazine Platinum containing Cisplatin compounds: Carboplatin Aroplatin Oxaliplatin Plant Alkaloids Vinca alkaloids: Vincristine Vinblastine Vindesine Vinorelbine Taxoids: Paclitaxel Docetaxel DNA Topoisomerase Inhibitors Epipodophyllins: Etoposide Teniposide Topotecan 9-aminocamptothecin Camptothecin Crisnatol mitomycins: Mitomycin C Anti-metabolites Anti-folates: DHFR inhibitors: Methotrexate Trimetrexate IMP dehydrogenase Mycophenolic acid Inhibitors: Tiazofurin Ribavirin EICAR Ribonuclotide reductase Hydroxyurea Inhibitors: Deferoxamine Pyrimidine analogs: Uracil analogs: 5-Fiuorouracil Floxuridine Doxifluridine Ratitrexed Cytosine analogs: Cytarabine (ara C) Cytosine arabinoside Fludarabine Purine analogs: Mercaptopurine Thioguanine DNA Antimetabolites: 3-HP 2′- deoxy-5-fluorouridine 5-HP alpha-TGDR aphidicolin glycinate ara-C 5-aza-2′-deoxycytidine beta-TGDR cyclocytidine guanazole inosine glycodialdehyde macebecin II pyrazoloimidazole Hormonal therapies: Receptor antagonists: Anti estrogen: Tamoxifen Raloxifene Megestrol LHRH agonists: Goserelin Leuprolide acetate Anti-androgens: Flutamide Bicalutamide Retinoids/Deltoids Cis-retinoic acid Vitamin A derivative: All-trans retinoic acid (ATRA-IV) Vitamin D3 analogs: EB 1089 CB 1093 KH 1060 Photodynamic therapies: Vertoporfin (BPD-MA) Phthalocyanine Photosensitizer Pc4 Demethoxy-hypocrellin A (2BA-2-DMHA) Cytokines: Interferon-.alpha. Interferon-.gamma. Tumor necrosis factor Angiogenesis Inhibitors: Angiostatin (plasminogen fragment) antiangiogenic antithrombin III Angiozyme ABT-627 Bay 12-9566 Benefin Bevacizumab BMS-275291 cartilage-derived inhibitor (CDI) CAI CD59 complement fragment CEP-7055 Col 3 Combretastatin A-4 Endostatin (collagen XVIII fragment) Fibronectin fragment Gro-beta Halofuginone Heparinases Heparin hexasaccharide fragment HMV833 Human chorionic gonadotropin (hCG) IM-862 Interferon alpha/beta/gamma Interferon inducible protein (IP-10) Interleukin-12 Kringle 5 (plasminogen fragment) Marimastat Metalloproteinase inhibitors (TIMPs) 2-Methoxyestradiol MMI 270 (CGS 27023A) MoAb IMC-1CIINeovastat NM-3 Panzem PI-88 Placental ribonuclease inhibitor Plasminogen activator inhibitor Platelet factor-4 (PF4) Prinomastat Prolactin 16 kD fragment Proliferin-related protein (PRP) PTK 787/ZK 222594 Retinoids Solimastat Squalamine SS 3304 SU 5416 SU6668 SU11248 Tetrahydrocortisol-5 tetrathiomolybdate thalidomide Thrombospondin-1(TSP-1) TNP-470 Transforming growth factor-beta (TGF-b) Vasculostatin Vasostatin (calreticulin fragment) ZD6126 ZD 6474 farnesyl transferase inhibitors (FTI) bisphosphonates Antimitotic agents: allocolchicine Halichondrin B colchicine colchicine derivative dolstatin 10 maytansine rhizoxin thiocolchicine trityl cysteine Others: Isoprenylation inhibitors: Dopaminergic neurotoxins: 1-methyl-4-phenylpyridinium ion Cell cycle inhibitors: Staurosporine Actinomycins: Actinomycin D Dactinomycin Bleomycins: Bleomycin A2 Bleomycin B2 Peplomycin Anthracyclines: Daunorubicin Doxorubicin (adriamycin) Idarubicin Epirubicin Pirarubicin Zorubicin Mitoxantrone MDR inhibitors: Verapamil Ca.sup.2+ATPase inhibitors: Thapsigargin. acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or riL2), interferon alfa-2a; interferon alfa-2b; interferon alfa-n1; interferon alfa-n3; interferon beta-1 a; interferon gamma-1 b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. 20-epi-1,25 dihydroxyvitamin 03; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotennustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; nneterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anti-cancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Means of utilizing the invention are applicable to existing cancer vaccines that utilize whole cells or lysates thereof in that existing cancer cell lines or vaccines can be modified to express endothelial antigens thus taking the shape of tumor-associated channels or tumor associated endothelium that is not derived from patient hematopoietic originating endothelial progenitor cells. Whole cancer cells may be allogeneic, syngeneic, or autologous to the treatment recipient. Typically they may be treated to make them proliferation incompetent by a technique which preserves preserve their immunogenicity and their metabolic activity. One typically used technique is irradiation. Such cells. Typically the same general type of tumor cell is used that the patient bears. For example, a patient suffering from melanoma will typically be administered proliferation incompetent melanoma cells. The cells may express and secrete a cytokine naturally or by transfection with a nucleic acid which directs such expression and secretion. One suitable cytokine is GM-CSF. For example, the tumor cell may express a transgene encoding GM-CSF as described in U.S. Pat. Nos. 5,637,483, 5,904,920, 6,277,368 and 6,350,445, as well as in U.S. Patent Publication No. 20100150946, each of which is expressly incorporated by reference. One example of a GM-CSF-expressing, genetically modified cancer cell for the treatment of pancreatic cancer is described in U.S. Pat. Nos. 6,033,674 and 5,985,290, both of which are expressly incorporated by reference herein. Other cytokines can be used. Suitable cytokines which may be used include cytokines which stimulate dendritic cell induction, recruitment, and/or maturation. Such cytokines include, but are not limited to, one or more of GM-CSF, CD40 ligand, IL-12, CCL3, CCL20, and CCL21. Granulocyte-macrophage colony stimulating factor (GM-CSF) polypeptide is a cytokine or fragment having immunomodulatory activity and having at least about 85% amino acid sequence identity to GenBank Accession No. AAA52122.1.

According to one alternative embodiment, cytokines are delivered by inactivated bystander cells which express and secrete one or more cytokines. The bystander cell may be a monocyte, a fibroblast, or a proliferating endothelial progenitor cell. Alternatively bystander cells may be mesenchymal stem cells transfected with cytokines or Type 1 mesenchymal stem cells. The bystander cells may provide all of the cytokines which stimulate dendritic cell induction, recruitment, and/or maturation, or may supplement cytokines secreted by the inactivated tumor cells. Immunomodulatory cytokine-expressing bystander cell lines are described in U.S. Pat. Nos. 6,464,973, and 8,012,469, Dessureault et al., Ann. Surg. Oneal. 14: 869-84, 2007, and Eager and Nemunaitis, Mol. Thor. 12: 18-27, 2005, each of which is expressly incorporated by reference.

In one embodiment the tumor vascular cells are fused with dendritic cells. For practice of the invention, both cell types must be isolated and electrofused together in a manner to allow viability and retained antigen presentation ability as described in prior works utilizing fusion between dendritic cells and tumor cells which are incorporated by reference [58, 59]. Specifically the antigen presenting machinery of the dendritic cell must be intact to present intracellular tumor derived antigens [60]. In one embodiment the fused cells are treated with agents that upregulate antigen processing machinery, agents such as valproic acid which increase expression of the transporter associated protein. The tumor vascular cells can be obtained from purification from a surgical specimen, short term cultured established tumor cell line, or allogeneic tumor cells which share antigens with the autologous tumor. The tissue is generally digested with collagenase and a suspension is purified from surgical material or from tissue culture. The cells in the tissue are subsequently coaxed to differentiate towards the endothelial lineage by treatment with endothelial media, or endothelial cytokines such as VEGF, IGF, EGF, FGF-1, FGF-2, FGF-5 or TGF. In one embodiment cells are first purified for expression of stem cell markers before treatment with endothelial differentiation media. In one embodiment, it is also necessary to generate human dendritic cells. Monocytes are isolated from peripheral blood mononuclear cells (PBMC) obtained from leukaphoresis [61]. Media containing Granular Monocytes- Colony Stimulating Factor (GM-CSF) and interleukin-4 (IL-4) are used for DC culture for seven days. During the last two days of the culture period, TNF-a and PGE2 are added. The recovered cells are dendritic cells that show a mature phenotype and express IL-12 [62-65]. The next step in the practice of this specific embodiment of the invention is to create fusion between the dendritic cells and tumor cells. The fusion media that may be used are of several types. One type of fusion media (Media A) can be created by combining approximately 5% glucose with 0.1 mM calcium acetate, 0.5 mM magnesium acetate, and 1% bovine BSA adjusted to a pH of 7.2. The pH can be adjusted by using histidine. An alternative type of media that can be used (Media B) is created by combining 5% glucose with 0.1 mM calcium acetate and 0.5 mM magnesium acetate without BSA. For induction of the fusion process, the ratio of dendritic cells to fusion cells should be about 1 to 2:1. For example, a 5 cc amount of fusion media would compel up to 75 million cells, with 50 million cells being dendritic cells and 25 million cells being tumor cells. The cells are aligned (dielectrophoresis) with alternative current at about 150 V/cm for 10 seconds. This is immediately followed by exposure of the cells to direct current of 1200V/em for 25 1 . . . 1 seconds. The direct current voltage is sometimes reduced by up to 20% depending on the tumor cell line. The cells are then diluted 1:10 in complete RPMI 1640 media with 10% human AB serum. All of the cells are cultured in a flask with RPMI1640 medium containing 10% FCS overnight (e.g., 12-18 hours). After the culture period, the flask is washed to remove any non-adherent cells (i.e., those cells not adhering to the walls of the flask). Adherent cells are those that compel DC-tumor fusion cells and, non-adherent cells are discarded because they do not compel DC-tumor cells, i.e., the non-adherent cells are mostly not fused and fused DCs. The adherent cells can be harvested by trypsin. The generated cells are subsequently administered to the patient at various concentrations sufficient to elicit immune response. In one embodiment cells are administered at approximately 1 million to 100 million per injection per month. In a preferred embodiment cells are administered at a concentration of approximately 10 million cells per injection.

According to another embodiment, the invention pertains to a method of conducting immunotherapy involving the administration of activated antigen presenting cells. In another embodiment, the invention involves the creation of antigen presenting cells (APCs) activated against cancer stem cells. As used herein, antigen presenting cells include but are not limited to dendritic cells, macrophages or natural killer cells. Other examples of cells that could serve as antigen presenting cells, include fibroblasts, glial cells and microglial cells.

In one embodiment of the invention, ascorbic acid is administered intravenously together with activated lymphocytes which possess tumor inhibitory/killing activity. In a preferred embodiment the intravenous vitamin C is administered once every two days at a concentration of 10 g per injection. The rational for use of intravenous vitamin C comes from observations of a scurvy-like condition in a renal cell carcinoma patient treated with IL-2. The patient presented with acute signs and symptoms of scurvy (perifollicular petechiae, erythema, gingivitis and bleeding). Serum ascorbate levels were significantly reduced to almost undetectable levels [66]. Although the role of ascorbic acid (AA) hypersupplementation in stimulation of immunity in healthy subjects is controversial, it is well established that AA deficiency is associated with impaired cell mediated immunity. This has been demonstrated in numerous studies showing deficiency suppresses T cytotoxic responses, delayed type hypersensitivity, and bacterial clearance [67]. Additionally, it is well-known that NK activity, which IL-2 is anti-tumor activity is highly dependent on, is suppressed during conditions of AA deficiency [68]. Thus it may be that while IL-2 therapy on the one hand is stimulating T and NK function, the systemic inflammatory syndrome-like effects of this treatment may actually be suppressed by induction of a negative feedback loop. Such a negative feedback loop with IL-2 therapy was successfully overcome by work using low dose histamine to inhibit IL-2 mediated immune suppression, which led to the “drug” Ceplene (histamine dichloride) receiving approval as an IL-2 adjuvant for treatment of AML [69].

In one embodiment of the invention, dendritic cells are activated against markers and antigens present in cancer stem cells esc through targeting the proximal cancer derived vascular cells, which typically are derived preferentially from cancer stem cells as compared to cancer non-stem cells. APCs are contacted with the marker or antigen, they are taken into the cell, processed and then presented on the surface of the cell. In another example, mRNA or DNA in CSCs is subjected to APCs, which also results in an activation against the CSCs from which the mRNA and/or DNA was procured. For stimulation of immunity tumor stem cells may be isolated by selection for markers associated with tumor stem cells such as CD133 or aldehyde dehydrogenase. In another example, dendritic cells are activated by fusion with a esc. The antigen presenting cells take in and digest the cancer stem cells by phagocytosis and/or endocytosis. Alternatively, or in conjunction with phagocytosis and/or endocytosis, the dendritic cells are subjected to electrical current in the presence of the CSCs. Means of electrofusion of cells are well known in the art, for which the following references are provided [70-81]. It is known in the art that the cellular products obtained following electrofusion (EF) of dendritic cells and tumour cells have shown promise as cancer vaccines. The immunogenicity of these preparations has been attributed to the presence of small numbers of DC-tumour hybrids and the contribution of the non-hybrid tumour cells present has received little attention. Optimized EF conditions to yield the maximum number of DC-SW620 hybrids co-expressing tumour associated antigen (TAA) and DC associated antigens. Exposure of SW620 to EF induced significant increases in apoptosis and necrosis. Pre-exposure of SW620 to the EF buffer alone [0.3 M glucose, 0.1 mM Ca(CH3C00)2 and 0.5 mM Mg(CH3C00)(2)] has been shown to result in significant increases in TAA uptake by DC during co-culture. In co-cultures of PBMC responders with SW620, the levels of IFNganrima release and cytotoxic activity were significantly increased by pre- exposure of the SW620 to EF. Pre-exposure of allogeneic non-T cells, the colorectal cell line Lovo and a breast cancer cell line (MCF7) to EF also significantly increased the levels of IFNganrinria release by responding PBMC. The methodology demonstrated by these authors is incorporated by reference [82], as useful for fusing of tumor vascular cells with dendritic cells for the purpose of generating an anticancer immunity.

In another embodiment, a tumor sample containing multiple cell types is procured from a subject. As has been discussed herein, it is the inventors' belief that if cancer stem cells can be preferentially targeted over other cells in a tumor this will dramatically improve cancer therapy. Accordingly, cancer stem cells are isolated or enriched from the tumor sample. Tumor samples may be procured from an allogeneic source, i.e., a subject of the same species but other than the subject into which activated antigen presenting cells are administered, as described in the reference attached utilizing dendritic cells together with osteosarcoma and incorporated by reference [83]. In other embodiments, the tumor samples are procured from an autologous source. For example, tumor cells are removed from a cancer subject, the cells are used to activate antigen presenting cells ex vivo and then the activated cells are administered to the cancer subject. Additionally, dendritic cell fusioned to tumor vascular cells may be stimulated to mature by addition of a toll like receptor agonist, in a manner similar to described in the presented reference [75]. Other maturation agents of dendritic cells such as CD40-CD40L [84] activation may be utilized. In one embodiment it is essentially that the lysosomes of the cells fused are intracellularly connected so as to allow for proper presentation of antigen [85].

Antibodies which are suitable for use in the treatment regimen and compositions and kits include any which specifically bind to Programmed Death 1. (PD-1). Exemplary types of antibodies which may be employed include without limitation human, humanized, chimeric, monoclonal, polyclonal, single chain, antibody binding fragments, and diabodies. Typically antibodies are substantially encoded by an immunoglobulin gene or immunoglobulin genes, or fragments thereof. Antibodies are capable of specifically binding an antigen or epitope. See, e.g. Fundamental Immunology, 3rd Edition, W. E. Paul, ed., Raven Press, N.Y. (1993); Wilson (1994; J. Immunol. Methods 175:267-273; Yarmush (1992) J. Biochem. Biophys. Methods 25:85-97. An antibody typically specifically binds to an antigen or epitope. Specific binding occurs to the corresponding antigen or epitope even in the presence of a heterogeneous population of proteins and other biologics. Specific binding of an antibody indicates that it binds to its target antigen or epitope with an affinity that is substantially greater than binding to irrelevant antigens The relative difference in affinity is often at least 25% greater, more often at least 50% greater, most often at least 100%. The relative difference can be at least 2.times., at least 5.times., at least 10.times., at least 25.times., at least 50.times., at least 100.times., at least 1.000.times., for example.

In order to stimulate immunogenicity, stimulators of toll like receptors may be added as part of a vaccination composition. Toll like receptors (TLR) are a family of proteins that sense a microbial product and/or initiates an adaptive immune response. TLR operate mechanistically through stimulating DC maturation. TLRs are conserved membrane spanning molecules containing an ectodomain of leucine-rich repeats, a transmembrane domain and an intracellular TIR. (Toii/IL-1R) domain. TLRs recognize distinct structures in microbes; often referred to as “PAMPs” (pathogen associated molecular patterns). Ligand binding to TLRs invokes a cascade of intra-cellular signaling pathways that induce the production of factors involved in inflammation and immunity. Numerous TLR agonists are known in the art, they include lipoproteins, lipopolypeptides, peptidoglycans, zymosan, lipopolysaccharide; neisserial porins, flagellin, profillin, galactoceramide, muramyl dipeptide,glucopyranosyllipid A (GLA), and resiquimod (R848). Peptidoglycans, lipoproteins, and lipoteichoic acids are cell wall components, of Gram-positive. Lipopolysaccharides are expressed by most bacteria. Flagellin is the structural component of bacterial flagella that is secreted by pathogenic and commensal bacterial. A Galactosylceramide (.alpha.-GaiCer) is an activator of natural killer T (NKT) cells. Muramyl dipeptide is a bioactive peptidoglycan motif common to all bacteria. Such agonists mediate innate immune activation via Toll-like Receptors. Pam3Cys, a TLR-1/2 agonist; CFA, a TLR-2 agonist; MALP2, a TLR-2 agonist; Pam2Cys, a TLR-2 agonist; FSL-1, a TLR-2 agonist; Hib-OMPC, a TLR-2 agonist; polyribosinic:polyribocytidic acid (Poly I:C), a TLR-3 agonist; polyadenosine-polyuridylic acid (poly AU), a TLR-3 agonist; Polyinosinic-Polycytidylic acid stabilized with poly-L-lysine and carboxymethylcellulose (Hiltonoi.RTM.), a TLR-3 agonist; monophosphoryllipid A (MPL), a TLR-4 agonist; LPS, a TLR-4 agonist; bacterial flagellin, a TLR-5 agonist; sialyi-Tn (STn), a carbohydrate associated with the MUCI mucin on a number of human cancer cells and a TLR-4 agonist; imiquimod, a TLR-7 agonist; resiquimod, a TLR-7/8 agonist; loxoribine, a TLR-7/8 agonist; and unmethylated CpG dinucleotide (CpG-ODN), a TLR-9 agonist.

Formulation of the whole cancer vascular cells with the TLR agonist is thought in the current invention efficacy. Formulations can be incubated together for periods of times such as ¼, ½, 1, 2, 3, 5, 10, 24 hours, at temperatures such as 4 degrees C. Alternatively, binding in the presence of a lipophilic agent or an emulsifying agent can be employed. Such agents are well known in the art.

In one embodiment in vitro generated lymphocytes against tumor vasculature are produced. Peripheral blood is extracted from a cancer patient and peripheral blood monoclear cells (PBMC) are isolated using the Ficoll Method. PBMC are subsequently resuspended in 10 ml STEM-34 media and allowed to adhere onto a plastic surface for 2-4 hours. The adherent cells are then cultured at 37° C. in STEM-34 media supplemented with 1,000 U/mL granulocyte-monocyte colony-stimulating factor and 500 Wm! IL-4 after non-adherent cells are removed by gentle washing in Hanks Buffered Saline Solution (HBSS). Half of the volume of the GM-CSF and IL-4 supplemented media is changed every other day. Immature DCs are harvested on day 7. In one embodiment said generated DC are used to stimulate T cell and NK cell tumoricidal activity in the presence of tumor vasculature cells generated according to the invention. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti- CD3 antibody is added, together with approximately 1 ug/m1 anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to said T cells and/or NK cells in order to endow cytotoxic activity towards tumor cells. In a particular embodiment, inhibitors of caspases are added in the culture so as to reduce rate of apoptosis off cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel.

In some embodiments, the culture of the cells is performed by starting with purified lymphocyte populations, which are then cultured with tumor vasculature cells. The step of separating the cell population and cell sub-population containing aT cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing aT cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the invention, T regulatory cells are depleted before initiation of the culture. Depletion off regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-.beta., IL-15, IL-7, IFN-.alpha., IL-12, CD4OL, and IL-27. From the viewpoint of enhancing cellular immunity, particularly suitably, IL-2, IFN-.gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIPI.alpha., MIPI.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule.

Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring aT cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects.

In one example, The SKMEL2 human melanoma cell line was purchased from ATCC and grown in Dulbecco's modified Eagles's medium (DMEM) supplemented with 10% fetal bovine serum. All cells were incubated at 37° C. and 5% CO₂, with regular mycoplasma contamination test. For induction of differentiation into endothelial-like cells, conditions resembling tumor microenvironment were simulated. Specifically, human recombinant HMGB-1 protein (10 μg/ml, Sigma-Aldrich) was applied to cells for 72 h. Incubation of the cells was performed under normoxic conditions, and hypoxic conditions (1% oxygen). Significantly increased expression of endothelial markers such as CD31 within the culture is observed. CD31 positive cells are selected from culture using magnetic activated sorting (MACS) according to the manufacturer's instructions and utilized as an antigenic source for vaccination of melanoma patients.

1. A method of treating cancer comprising the steps of:
a) obtaining a tumor cell line;
b) treating said tumor cell line with agents and conditions recapitulating a tumor microenvironment in vitro;
c) isolating cells possessing properties of endothelial cells or tumor vascular channel cells; and
d) using said cells from step “c” as a source of immunogens for the purposes of vaccination.

2. The method of claim 1, wherein said tumor cell line is selected from a group comprised of: J82, RT4, ScaBER, T24, TCCSUP, 5637 Carcinoma, SK-N-MC Neuroblastoma, SK-N-SH Neuroblastoma, SW 1088 Astrocytoma, SW 1783 Astrocytoma, U-87 MG Glioblastoma, astrocytoma, grade III, U-118 MG Glioblastoma, U-138 MG Glioblastoma, U-373 MG Glioblastoma, astrocytoma, grade III, Y79 Retinoblastoma, BT-20 Carcinoma, breast, BT-474 Ductal carcinoma, breast, MCF7 Breast adenocarcinoma, pleural effusion, MDA-MB-134-V Breast, ductal carcinoma, pleural I effusion, MDA-MD-157 Breast medulla, carcinoma, pleural effusion, MDA-MB-175-VII Breast, ductal carcinoma, pleural Effusion, MDA-MB-361Adenocarcinoma, breast, metastasis to brain, SK-BR-3 Adenocarcinoma, breast, malignant pleural effusion, C-33 A Carcinoma, cervix, HT-3 Carcinoma, cervix, metastasis to lymph node ME-180 Epidermoid carcinoma, cervix, metastasis to omentum, MEL-175 Melanoma, MEL-290 Melanoma, HLA-A*0201Melanoma cells, MS751Epidermoid carcinoma, cervix, metastasis to lymph Node, SiHa Squamous carcinoma, cervix, JEG-3 Choriocarcinoma, Caco-2 Adenocarcinoma, colon HT-29 Adenocarcinoma, colon, moderately well-differentiated grade II, SK-CO-1Adenocarcinoma, colon, ascites, HuTu 80 Adenocarcinoma, duodenum, A-253 Epidermoid carcinoma, submaxillary gland FaDu Squamous cell carcinoma, pharynx, A-498 Carcinoma, kidney, A-704 Adenocarcinoma, kidney Caki-1 Clear cell carcinoma, consistent with renal primary, metastasis to skin, Caki-2 Clear cell carcinoma, consistent with renal primary, SK-NEP-1Wilms' tumor, pleural effusion, SW 839 Adenocarcinoma, kidney, SK-HEP-1Adenocarcinoma, liver, ascites, A-427 Carcinoma, lung Calu-1Epidermoid carcinoma grade III, lung, metastasis to pleura, Calu-3 Adenocarcinoma, lung, pleural effusion, Calu-6 Anaplastic carcinoma, probably lung, SK-LU-1Adenocarcinoma, lung consistent with poorly differentiated, grade III, SK-MES-1Squamous carcinoma, lung, pleural effusion, SW 900 Squamous cell carcinoma, lung, EBIBurkitt lymphoma, upper maxilia, EB2 Burkitt lymphoma, ovary P3HR-1Burkift lymphoma, ascites, HT-144 Malignant melanoma, metastasis to subcutaneous tissue Malme-3M Malignant melanoma, metastasis to lung, RPMI-7951 Malignant melanoma, metastasis to lymph node, SK-MEL-1 Malignant melanoma, metastasis to lymphatic system, SK-MEL-2 Malignant melanoma, metastasis to skin of thigh, SK-MEL-3 Malignant melanoma, metastasis to lymph node SK-MEL-5 Malignant melanoma, metastasis to axillary node, SK-MEL-24 Malignant melanoma, metastasis to node, SK-MEL-28 Malignant melanoma, SK-MEL-31 Malignant melanoma, Caov-3 Adenocarcinoma, ovary, consistent with primary, Caov-4 Adenocarcinoma, ovary, metastasis to subserosa of fallopian tube, SK-OV-3 Adenocarcinoma, ovary, malignant ascites, SW 626 Adenocarcinoma, ovary, Capan-1Adenocarcinoma, pancreas, metastasis to liver, Capan-2 Adenocarcinoma, pancreas, DU 145 Carcinoma, prostate, metastasis to brain, A 204 Rhabdomyosarcoma, Saos-2 Osteogenic sarcoma, primary, SK-ES 1 Anaplastic osteosarcoma versus Swing sarcoma, SK-LNS-1Leiomyosarcoma, vulva, primary, SW 684 Fibrosarcoma, SW 872 Liposarcoma SW 982 Axilla synovial sarcoma, SW 1353 Chondrosarcoma, humerus, U-2 OS Osteogenic sarcoma, bone primary, Malme-3 Skin fibroblast, KATO III Gastric carcinoma, Cate-IB Embryonal carcinoma, testis, metastasis to lymph node, Tera-1 Embryonal carcinoma, Tera-2 Embryonal carcinoma, SW579 Thyroid carcinoma, AN3 CA Endometrial adenocarcinoma, metastatic, HEC-I-A Endometrial adenocarcinoma HEC-1-B Endometrial adenocarcinoma, SK-UT-1 Uterine, mixed mesodermal tumor, consistent with Ieiomyosarcomagrade III, SK-UT-IB Uterine, mixed mesodermal tumor, Sk-Me128 Melanoma SW 954 Squamous cell carcinoma, vulva, SW 962 Carcinoma, vulva, lymph node metastasis, NCI-H69 Small cell carcinoma, lung, NCI-H128 Small cell carcinoma, lung, BT-483 Ductal carcinoma, breast BT-549 Ductal carcinoma, breast, DU4475 Metastatic cutaneous nodule, breast carcinoma HBL-100 Breast, Hs 578Bst Breast, Hs 578T Ductal carcinoma, breast, MDA-MB-330 Carcinoma, breast MDA-MB-415 Adenocarcinoma, breast, MDA-MB-435s Ductal carcinoma, breast, MDA-MB-436 Adenocarcinoma, breast, MDA-MB-453 Carcinoma, breast, MDA-MB-468 Adenocarcinoma, breast T-47D Ductal carcinoma, breast, pleural effusion, Hs 766T Carcinoma, pancreas, metastatic to lymph node, Hs 746T Carcinoma, stomach, metastatic to left leg, Hs 695T Amelanotic melanoma, metastatic to lymph node, Hs 683 Glioma, Hs 294T Melanoma, metastatic to lymph node, Hs 602 Lymphoma, cervical JAR Choriocarcinoma, placenta, Hs 445 Lymphoid, Hodgkin's disease, Hs 700T Adenocarcinoma, metastatic to pelvis, H4 Neuroglioma, brain, Hs 696 Adenocarcinoma primary, unknown, metastatic to bone-sacrum, Hs 913T Fibrosarcoma, metastatic to lung, Hs 729 Rhabdomyosarcoma, left leg, FHs 738Lu Lung, normal fetus, FHs 173We Whole embryo, normal, FHs 738B1 Bladder, normal fetus NIH:OVCAR-3 Ovary, adenocarcinoma, Hs 67 Thymus, normal, RD-ES Ewing's sarcoma ChaGo K-1Bronchogenic carcinoma, subcutaneous, metastasis, human, WERI-Rb-1Retinoblastoma NCI-H446 Small cell carcinoma, lung, NCI-H209 Small cell carcinoma, lung, NCI-H146 Small cell carcinoma, lung, NCI-H441Papillary adenocarcinoma, lung, NCI-H82 Small cell carcinoma, lung H9 T-celllymphoma, NCI-H460 Large cell carcinoma, lung, NCI-H596 Adenosquamous carcinoma, lung NCI-H676B Adenocarcinoma, lung, NCI-H345 Small cell carcinoma, lung, NCI-H820 Papillary adenocarcinoma, lung, NCI-H520 Squamous cell carcinoma, lung, NCI-H661Large cell carcinoma, lung NCI-H510A Small cell carcinoma, extra-pulmonary origin, metastatic D283 Med Medulloblastoma Daoy Medulloblastoma, D341Med Medulloblastoma, AML-193 Acute monocyte leukemia MV4-11 Leukemia biphenotype.

3. The method of claim 1, wherein said tumor cell line is generated from a patient de novo.

4. The method of claim 1, wherein said tumor cell line comprises tissue derived from primary tumor sources.

5. The method of claim 1, wherein said agents recapitulating the tumor microenvironment are selected from a group comprising:
a) angiopoietin;
b) EGF;
c) TGF-beta;
d) PGE-2;
e) FGF-1;
f) FGF-2;
g) FGF-5;
h) IGF-1;
i) HGF; and
j) hCG.

6. The method of claim 1, wherein said conditions recapitulating the tumor microenvironment are selected from a group comprising:
a) increased acidity;
b) hypoxia;
c) three-dimensional culture; and
d) presence of inflammatory cells.

7. The method of claim 6, wherein said inflammatory cells are selected from a group comprising:
a) monocytes;
b) neutrophils;
c) basophils;
d) eosinophils;
e) mast cells; and
f) mesenchymal stem cells.

8. The method of claim 7, wherein said monocytes are differentiated into M2 lineage.

9. The method of claim 8, wherein said monocytic differentiation into the M2 lineage is accomplished by treatment with IL-4 and/or IL-13.
